(19) United States
(12) Patent Application Publication
(10) Pub. No.: US 2021/0220320 A1
(43) Pub. Date: Jul. 22, 2021

(54) ANALGESIC AGENT AND SEDATIVE AGENT

(71) Applicant: THE UNIVERSITY OF TOKYO, Tokyo (JP)

(72) Inventors: Yuya TERASHIMA, Tokyo (JP); Etsuko TODA, Tokyo (JP); Kouji MATSUSHIMA, Tokyo (JP)

(73) Assignee: THE UNIVERSITY OF TOKYO, Tokyo (JP)

(21) Appl. No.: 16/966,263

(22) PCT Filed: Jan. 31, 2019
(86) PCT No.: PCT/JP2019/003418

§ 371 Date: Apr. 6, 2021

(30) Foreign Application Priority Data
Jan. 31, 2018 (JP) 2018-015635

(51) Int. Cl. A61K 31/325; A61K 31/145; A61P 25/02; A61P 25/04

(52) U.S. Cl. CPC A61K 31/325; A61P 25/04; A61P 25/02; A61K 31/145

(57) ABSTRACT

Disclosed is a novel means that is effective for analgesia or sedation. The analgesic or sedative agent of the present invention comprises, an active component, disulfiram, a metal complex of diethyldithiocarbamate, a disulfide capable of generating diethyldithiocarbamate in the body, a pharmaceutically acceptable salt of any of these compounds, or a solvate of any of these compounds or salts thereof. Disulfiram and the above-described metal complex are known to have anticancer and anti-inflammatory effects. The agent of the present invention can exert multiple therapeutic benefits such as anticancer, anti-inflammatory, analgesic, and sedative effects, in cancer patients.

### TECHNICAL FIELD

The present invention relates to an analgesic agent and a sedative agent.

### BACKGROUND ART

Analgesic drugs are roughly divided into opioid and non-opioid analgesics. Non-opioid analgesics include nonsteroidal anti-inflammatory drugs (NSAIDs) such as aspirin, loxoprofen, and indometacin, and acetaminophen, which lacks anti-inflammatory effects. Since induction of inflammation leads to production of analgesic substances such as bradykinin, many of the anti-inflammatory agents also exert their effects as antiphlogistic analgesic agents. Indeed, NSAIDs are sold as over-the-counter analgesic drugs, as well as are used as prescription drugs.

Adequate control of pain in, for example, cancer or inflammatory diseases is very important. It can be difficult to control pain in diseases associated with inflammation only by means of an agent with anti-inflammatory effects. Therefore, opioid analgesics, which are narcotic analgesic drugs with strong analgesic effects, are sometimes prescribed for severe pain, such as pain in advanced cancer. Pain is classified as nociceptive pain, neuropathic pain, or psychogenic pain, based on the pain mechanisms. Any drug, such as NSAIDs or opioid analgesic drugs, is used in a graded manner to treat a pain condition, depending on the mechanism and/or severity of the pain, but the pathogenic mechanism of the pain may be a combination of multiple pain mechanisms, which causes many conditions where pain control is difficult. Additionally, when those drugs are used in combination, special attention should be paid to the side effect problem and limited effect of each drug. In particular, existing analgesic drugs are hard to exhibit their effects on one of the pain mechanisms, neuropathic pain, which leads to a need for development of analgesic drugs based on a novel mechanism of action.

Disulfiram has an aldehyde dehydrogenase-inhibiting activity, and inhibits ethanol metabolism in the liver to cause accumulation of acetaldehyde in the body, which is responsible for hangover symptoms. Thus, after taking disulfiram, hangover symptoms occur even with a small amount of alcohol. By utilizing this action, disulfiram is used as an anti-alcoholism drug for treatment of chronic alcoholism. In addition to this action, disulfiram is known to exhibit anticancer effects by its action to induce death of cancer cells or cancer stem cells per se (for example, Non-Patent Documents 1 to 3 and Patent Document 1), and further known to exhibit anticancer and anti-inflammatory effects by controlling cells which constitute cancer and inflammatory microenvironments (Patent Document 2).

However, the fact that disulfiram has analgesic effects has not at all been known. Patent Document 3 reports the analgesic effect of diethylamine, which is a metabolite of disulfiram, but this document also reports that disulfiram has no analgesic effects. Moreover, the package insert (Non-Patent Document 4) for an anti-alcoholism drug “NOCBIN,” which contains disulfiram as an active component, describes the possible development of headache or joint pain as side effects, but does not describe any action related to analgesic effects. The fact that disulfiram has sedative effects has also not at all been known.

### PRIOR ART DOCUMENTS

### Patent Documents

Patent Document 1: JP 2013-100268 A
Patent Document 2: WO 2016/111307
Patent Document 3: WO 2008/075993

### Non-Patent Documents

Non-Patent Document 1: Cancer Research, Vol. 66, pp. 10425-10433 (2006)
Non-Patent Document 2: Clinical Cancer Research, Vol. 15, pp. 6070-6078 (2009)
Non-Patent Document 3: Molecular Cancer Therapeutics, Vol. 1, pp. 197-204 (2002)
Non-Patent Document 4: The package insert for “NOCBIN Powder”, 10th edition, revised in April 2015 (authorization number: 22000AMX02130)

### SUMMARY OF THE INVENTION

### Problems to be Solved by the Invention

An object of the present invention is to provide a novel means that is effective for analgesia or sedation.

### Means for Solving the Problems

The inventors studied hard and consequently found that disulfiram surprisingly has stronger analgesic effects than those of existing analgesic agents, and that a metal complex of diethyldithiocarbamate, which is a metabolite of disulfiram, also has strong analgesic effects, and furthermore that disulfiram and a metal complex of diethyldithiocarbamate have sedative effects as well, and then completed the present invention.

That is, the present invention provides an analgesic or sedative agent comprising any of the following (1) to (5) as an active component:

(1) disulfiram;
(2) a metal complex of diethyldithiocarbamate;
(3) a disulfide capable of generating diethyldithiocarbamate in the body;
(4) a pharmaceutically acceptable salt of (1), (2), or (3); and
(5) a solvate of (1), (2), (3), or (4).

### Effect of the Invention

By the present invention, a novel agent with strong analgesic effects was provided. Disulfiram is an agent that has been used as an anti-alcoholism drug in Japan since 1983, and its long-term safety has been established. Therefore, disulfiram can be widely used for treating various levels of pain, including mild to moderate pain such as headache or stomachache, for which NSAIDs are conventionally prescribed, and also strong pain which requires administration of a prescription drug. Pregabalin is a pain therapeutic that has been approved for use for neuropathic pain and fibromyalgia-related pain and whose analgesic effect is particularly strong among those of existing analgesic agents, but the analgesic agent according to the present invention exhibits a stronger pain-inhibiting effect than pregabalin.

It is also known that disulfiram and a metal complex of diethyldithiocarbamate, which is a metabolite of disulfiram, have anticancer and anti-inflammatory effects. In addition, diethyldithiocarbamate itself is known to have anticancer and anti-inflammatory effects (Patent Document 2), and therefore a disulfide capable of generating diethyldithiocarbamate in the body is likewise considered to have anticancer and anti-inflammatory effects. Thus, the agent of the present invention can be used to treat cancer or inflammation and simultaneously to treat and relieve pain in patients suffering from cancer or inflammatory diseases. There are very few pain therapeutics that exhibit such an additive drug effect in cancer therapy. In cancer therapy, μ-opioid analgesics such as morphine are often used but carry a high risk of side effects for patients since p-opioid analgesics are narcotics and/or controlled drugs and, additionally, cause severe constipation as a side effect through inhibition of intestinal movement and, furthermore, have a respiratory depression effect; hence, p-opioid analgesics should be used carefully. In consideration of such risks, the agent of the present invention is useful enough for pain control particularly in cancer therapy. Even when a p-opioid analgesic such as morphine is used, the agent of the present invention is additionally used to reduce the amount of the p-opioid analgesic used, which is in turn expected to reduce the above-described risks inherent to the p-opioid analgesic. Moreover, the agent of the present invention also exhibits sedative effects. Thus, the agent of the present invention is expected to be able to exert multiple therapeutic benefits such as anticancer, anti-inflammatory, analgesic, and sedative effects, particularly in cancer patients.

### BRIEF DESCRIPTION OF THE DRAWINGS

FIG. 1A shows an example of results of measurement of pain-like behavior in mice subjected to the formalin test. An agent (Comp) and a solvent (Vehicle) were administered intraperitoneally to mice in a drug administration group and a control group, respectively, and 30 minutes later, formalin was administered subcutaneously into the palmar surface of a paw in each mouse.

FIG. 1B shows pain scores of mice in a disulfiram (DSF)-administration group and a control group. * indicates a significant difference at p<0.05, and *** indicates a significant difference at p<0.001 (Student's t-test).

FIG. 1C shows the pain-inhibiting effect and its dose dependency assessed in each disulfiram (DSF) dose group. The pain score was measured in each DSF dose group, and the pain-inhibiting effect in each group was represented with the pain score of a control group defined as 100%. * indicates a significant difference at p<0.05, ** indicates a significant difference at p<0.01, and *** indicates a significant difference at p<0.001, from the control group (Student's t-test).

FIG. 1D shows a graph depicting the pain-inhibiting effect of DSF compared to those of existing analgesic agents (pregabalin, loxoprofen, and aspirin). The pain score in each drug administration group was measured, and the pain-inhibiting effect of each agent was represented with the pain score of a control group defined as 100%. * indicates a significant difference at p<0.05, ** indicates a significant difference at p<0.01, and *** indicates a significant difference at p<0.001, from the control group (Student's t-test).

FIG. 2 shows results of an evaluation of the analgesic effect of disulfiram in a sciatic nerve ligation (CCI) model. Sham Cont represents sham operation group; CCI Cont represents CCI-treated non-DSF administration group; CCI DSF represents CCI-treated DSF administration group. ** indicates a significant difference at p<0.01 (Student's t-test).

FIG. 3 shows pain-inhibiting effects of copper-diethyldithiocarbamate complex (Cu(DDC)₂) and iron(III)-diethyldithiocarbamate complex (Fe(DDC)₃) evaluated by means of the formalin test. * indicates a significant difference at p<0.05 from the control group (Student's t-test).

### MODE FOR CARRYING OUT THE INVENTION

The analgesic or sedative agent of the present invention comprises any of the following (1) to (5) as an active component.

(1) disulfiram.
(2) a metal complex of diethyldithiocarbamate.
(3) a disulfide capable of generating diethyldithiocarbamate in the body.
(4) a pharmaceutically acceptable salt of (1), (2), or (3).
(5) a solvate of (1), (2), (3), or (4).

In one aspect, the agent of the present invention comprises disulfiram, a pharmaceutically acceptable salt of disulfiram, or a solvate of disulfiram or the pharmaceutically acceptable salt thereof as an active component. In another aspect, the agent of the present invention comprises a metal complex of diethyldithiocarbamate, a pharmaceutically acceptable salt of the complex, or a solvate of the complex or the pharmaceutically acceptable salt thereof as an active component.

Disulfiram (chemical name: tetraethylthiuram disulfide) described as (1) above itself is a known compound, and is conventionally used as an anti-alcoholism drug for treatment of chronic alcoholism. Disulfiram is a prescription drug listed in the Japanese Pharmacopoeia, and its production method is well known.

The metal complex of diethyldithiocarbamate (DDC) described as (2) above may be a complex of any metal. The metal may be a monovalent, divalent, or higher valent metal. In one aspect, the metal complex of diethyldithiocarbamate is a complex of a divalent or higher valent metal. Specific examples of the metal complex include, but are not limited to, monovalent metal complexes, such as sodium complex and lithium complex; and complexes of divalent or higher valent metals, such as copper complex, iron(II) complex, iron(III) complex, zinc complex, platinum complex, gold complex, aluminium complex, magnesium complex, vanadium complex, selenium complex, cobalt(II) complex, and cobalt(II) complex.

Typically, the disulfide described as (3) above is a compound that produces at least one DDC molecule, for example one DDC molecule, in the body through cleavage of S—S bond in one molecule of the disulfide compound. Such a disulfide compound may be a compound which has a structure represented by Formula 1 below in its molecule. The wavy line in Formula 1 indicates that the remaining moiety beyond this line is omitted. The remaining moiety beyond the wavy line may be any structure, as long as the structure does not disturb the cleavage of S—S bond.

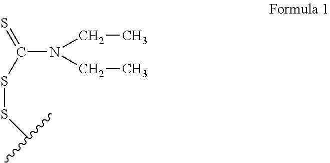 [SMILES: *SSC(=S)N(CC)CC]

Specific examples of the disulfide described as (3) above include, but are not limited to DDC-added disulfide compounds formed via SS exchange reaction between disulfide compounds, such as oxidized glutathione (GSSG: Glutathione-S—S-Glutathione), and DSF (i.e., a compound represented by Formula 1 in which the structure beyond the wavy line is glutathione), and DDC-added proteins formed via SS exchange reaction between proteins having at least a pair of functional cysteine residues, such as thioredoxin, and DSF (for example, a compound represented by Formula 1 in which the structure beyond the wavy line is thioredoxin).

Each of the compounds (1) to (3) may be used in the form of a pharmaceutically acceptable salt thereof. The salt may be an acid addition salt or a base addition salt. Specific examples of the acid addition salt include inorganic acid salts such as hydrochloric acid salt, hydrobromic acid salt, sulfuric acid salt, hydroiodic acid salt, nitric acid salt, and phosphoric acid salt; and organic acid salts such as citric acid salt, oxalic acid salt, acetic acid salt, formic acid salt, propionic acid salt, benzoic acid salt, trifluoroacetic acid salt, maleic acid salt, tartaric acid salt, methanesulfonic acid salt, benzenesulfonic acid salt, and para-toluenesulfonic acid salt. Specific examples of the base addition salt include inorganic base salts such as sodium salt, potassium salt, calcium salt, magnesium salt, and ammonium salt; and organic base salts such as triethylammonium salt, triethanolammonium salt, pyridinium salt, and diisopropylammonium salt. Any of the salts can be produced by any method known in the art of chemical synthesis.

Additionally, each of the compounds (1) to (3) and pharmaceutically acceptable salts thereof may also be used in the form of a solvate. Specific examples of the solvate include hydrates and ethanolates, but the solvate is not limited thereto and may be any solvate as long as it is a solvate with a pharmaceutically acceptable solvent. The solvates of the compounds (1) to (3) and salts thereof can be produced by any method known in the art of chemical synthesis.

In general, pain is classified according to the etiology into nociceptive pain, neuropathic pain (neurogenic pain), and psychogenic pain. Nociceptive pain is further classified into somatic pain and visceral pain. Neuropathic pain is classified into peripheral neuropathic pain and central neuropathic pain, according to the location of a nerve injury. Pain is also classified according to the duration into chronic pain and acute pain. Types of pain for which the analgesic agent of the present invention is used is not specifically limited, and the analgesic agent of the present invention can be used for any of the above-described types of pain.

Specific examples of the pain for which the analgesic agent of the present invention is administered include, for example, cancer pain; herpetic pain; postherpetic neuralgia; trigeminal neuralgia; diabetic neuropathic pain; pain due to, for example, phantom limb pain; headache; stomachache; low back pain; chronic pelvic pain syndrome; bladder pain; toothache; pain associated with various inflammatory diseases; pain due to, for example, surgery or trauma; and pain associated with gynecologic diseases including endometriosis, uterine myoma, and dysmenorrhea.

Cancer pain is one of the specific examples of pain for which the analgesic agent of the present invention is administered. Cancer pain includes somatic pain (for example, local pain from bone metastasis, wound pain early in the postoperative course, and pain associated with fascia or skeletal muscle inflammation), visceral pain (for example, abdominal pain associated with gastrointestinal obstruction, upper abdomen and flank pain associated with intratumoral hemorrhage in the liver, and upper abdomen and back pain associated with pancreatic cancer), neuropathic pain (for example, pain with numbness in the upper limb associated with brachial plexus infiltration of cancer, back pain associated with epidural infiltration of the vertebral metastasis or with spinal cord compression syndrome, and hand and foot pain after chemotherapy), and referred pain, which is classified into somatic pain or visceral pain and is perceived at a location adjacent or distant from the site of lesion (see Clinical Guidelines for Cancer Pain Management 2014, Japanese Society for Palliative Medicine, eds.). Furthermore, in addition to these pains caused by cancer itself, the analgesic agent of the present invention can also be suitably used for cancer-related pain, which is indirectly caused by cancer (for example, pain from bedsores, which happen when a patient is bedridden for a long period of time), pain related to cancer therapy (for example, pain due to postoperative scar or nerve injury, stomatitis caused by anticancer drug therapy, and skin burn caused by radiation therapy), pain which a patient originally suffers from and is unrelated to cancer, such as headache, and pain which occurs as a complication of cancer.

As described in Examples below, disulfiram and a metal complex of diethyldithiocarbamate also have sedative effects, and thus the compound (3) is likewise considered to have sedative effects. Therefore, any of the above-described compounds (1) to (5) can also be used as a sedative agent. The agent of the present invention can exert multiple therapeutic benefits such as anticancer, anti-inflammatory, analgesic, and sedative effects, in cancer patients.

The route of administration of the agent according to the present invention is not specifically limited, and may be systemic or local administration, and may be oral or parenteral administration. Examples of the parenteral administration include intramuscular administration, subcutaneous administration, intravenous administration, intraarterial administration, and transdermal administration.

The dosage form of the agent according to the present invention is also not specifically limited, and can be prepared by combining the active component such as disulfiram with additives suitable for each administration route as appropriate, such as pharmaceutically acceptable carrier, diluent, excipient, binder, lubricant, disintegrating agent, sweetener, suspending agent, emulsifier, coloring agent, taste masking agent, and stabilizing agent. Examples of the formulation include oral preparations such as tablets, hard capsules, soft capsules, granules, powders, and syrups; and parenteral preparations such as inhalants, injections, suppositories, and solutions. Formulation methods and additives which can be used are well known in the field of pharmaceutical formulation, and any of the methods and additives can be used.

Techniques for preparing sustained release formulations are also well known. The agent of the present invention may be provided as a sustained release formulation to stabilize and maintain the blood level of the active component. The term “sustained release” used herein has the same meaning as controlled release, and also includes delayed release and the like. The techniques for preparing sustained release formulations are classified into the single-unit type and the multiple-unit type based on the form of the sustained release formulation, or classified into the reservoir type, matrix type and the like based on the release control mechanism. Hybrid types, in which multiple mechanisms are combined, are also known. When the agent of the present invention is prepared as a sustained release formulation, any of the techniques for preparing sustained release formulations may be used. A DDS such as liposomes may also be used for the preparation. The sustained release formulation may be prepared into any dosage form, including tablet, granule, capsule, or the like. Specific examples of the sustained release formulation of disulfiram include the disulfiram formulation described in WO 2012/076897 A1, in which liposomes are used as a DDS, and the solid dispersion tablets of disulfiram described in International Journal of Pharmaceutics 497 (2016) 3-11, in which a polyvinyl acetate-polyvinyl pyrrolidone mixture or hypromellose is used as a sustained release polymer. However, the sustained release formulation of disulfiram is not limited to these specific examples.

The amount of the agent according to the present invention to be administered is appropriately determined depending on the conditions and body weight of a patient, administration route, and the like. For example, about 0.001 mg to about 20 g, e.g. about 0.1 mg to about 5 g, of the active component may be administered to an adult human (with a body weight of about 60 kg) per day in a single dose or in several divided doses, but the amount of the active component to be administered is not limited thereto.

Subjects to which the agent of the present invention is administered are typically mammals, and the agent of the present invention can be used for various mammals, including human, mouse, rat, hamster, rabbit, cat, dog, cow, horse, sheep, monkey, and the like.

The agent of the present invention may be used in combination with any existing analgesic drug. Examples of the existing analgesic drug which can be used in combination with the agent of the present invention include, but are not limited to, non-opioid analgesics (NSAIDs such as aspirin, loxoprofen and indometacin, and acetaminophen), opioid analgesics (such as morphine, fentanyl, oxycodone, codeine, tramadol, pentazocine, and buprenorphine), as well as antidepressant drugs such as duloxetine and anticonvulsant drugs such as gabapentin, pregabalin and carbamazepine, which have been used in Japan and other countries as therapeutic agents for nociceptive pain, fibromyalgia-related pain or trigeminal neuralgia and called adjuvant analgesics in the field of cancer pain management. An analgesic drug with stronger analgesic effects is more prone to produce severe side effects. By using the agent of the present invention in combination therewith, the amount of the existing analgesic drug used can be reduced, which is in turn expected to reduce the side effects.

### Examples

The present invention is described below by way of Examples more concretely. However, the present invention is not limited to the Examples described below.

### 1. Evaluation of Analgesic Effects of Disulfiram by Formalin Test

The formalin test, which is an inflammatory pain model and is commonly used as a neuropathic pain model in a broad sense, was used to evaluate the pain-inhibiting effect of disulfiram (DSF).

### <Methods>

In the formalin test, each mouse was placed in a transparent container for 30 minutes for acclimation to the analysis environment. Thereafter, a test drug or a solvent control (a solution of 10% DMSO and 2% Tween-80) was administered intraperitoneally to each mouse, and 30 minutes later, 20 μL of 1% formalin was injected subcutaneously from the palmar surface of the right hind paw to observe pain behavior in each mouse. The duration (sec.) of paw lifting and paw licking was used as a measured value of intensity of pain.

### <Results>

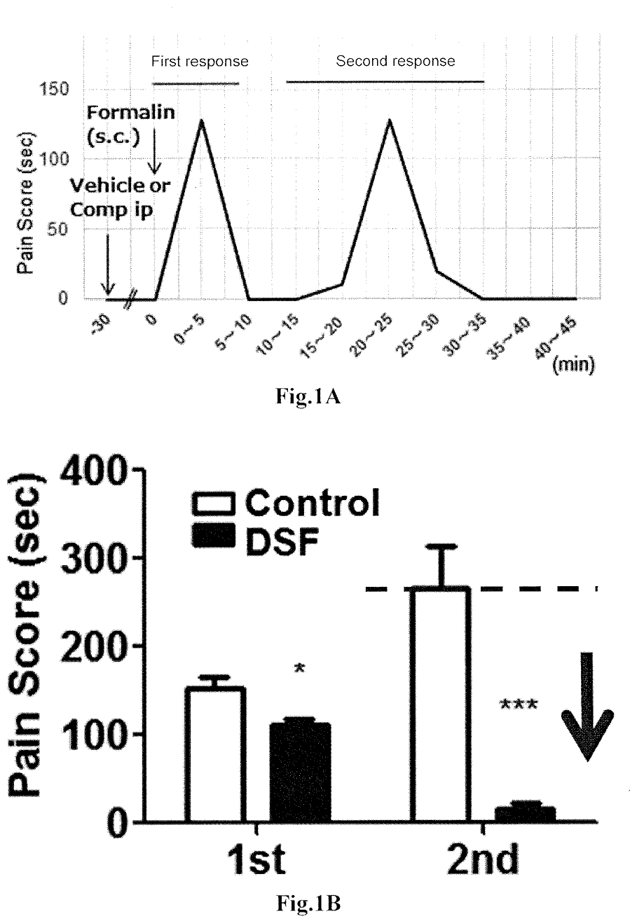

The formalin test is one of the commonly used models that are used as pain models to evaluate analgesic effects. A biphasic pain-like behavior is observed in the formalin test; the first response appears within 5 minutes and disappears till 10 minutes after the administration of formalin, and the second response is a response that reappears within 10 to 15 minutes after the administration of formalin (FIG. 1A). Administration of DSF at a dose of 40 mg/kg (equivalent to the dose of DSF commonly used as an anti-alcoholism drug) strongly suppressed the second response (FIG. 1B; *** indicates a significant difference at p<0.001 (Student's t-test)). The level of suppression of the second response in each DSF dose group was relatively evaluated by defining the level of the second response in the solvent control (a solution of 10% DMSO and 2% Tween-80) administration group as 100% to find that DSF exhibited analgesic effects in a dose-dependent manner and that administration of DSF even at a dose of 10 mg/kg resulted in significant analgesic effects (FIG. 1C; * indicates a significant difference at p<0.05. * indicates a significant difference at p<0.01, and *** indicates a significant difference at p<0.001, from the solvent control administration group). We also found that mice receiving administration of DSF behaved more quietly, indicating the sedative effect of DSF.

For the purpose of comparing the activity of DSF with those of existing analgesics such as pregabalin, the level of suppression of the second response in each drug administration group was evaluated in a relative manner, revealing that DSF had stronger analgesic effects than all of the tested existing analgesics under the condition where the drugs were administered at a fixed dose of 40 mg/kg (FIG. 1D). These results indicated that DSF, which had not been reported to have any pain-inhibiting effect, had strong analgesic effects.

### 2. Evaluation of Analgesic Effects of Disulfiram in Sciatic Nerve Ligation Model

Since central sensitization was known to be involved in the second phase pain response in the formalin test, DSF was expected to also exert its effects on neuropathic pain. However, the formalin test is a pain assessment model in which a stimulant-induced pain response associated with stimulant administration is assessed, which may be far different from a condition where pain is elicited by tactile sensation and the like that do not normally elicit pain (allodynia) or from clinical neuropathic pain that induces spontaneous pain. Thus, the sciatic nerve ligation (CCI) model, which is commonly used as a neuropathic pain mouse model, was used to evaluate the efficacy of DSF.

In the sciatic nerve ligation (CCI) mouse group, a femoral region of each mouse was cut open and the muscle layer was removed to expose the sciatic nerve, and the sciatic nerve was ligated with 6-0 silk sutures at three positions spaced 1 mm apart, and the incision was then closed. In contrast, each mouse in the sham operation (Sham) group was subjected to the same operation till the muscle layer was removed, and the incision was then closed without ligating the exposed sciatic nerve. On one week postsurgery or later, the planter test was performed to evaluate the pain threshold for heat, which was a stimulus not associated with stimulant administration, in those mice. DSF was administered intraperitoneally to mice in the CCI DSF group at a dose of 40 mg/kg. The solvent control (a solution of 10% DMSO and 2% Tween-80) was administered instead of DSF to mice in the CCI control group. The plantar test was performed 30 minutes after administration.

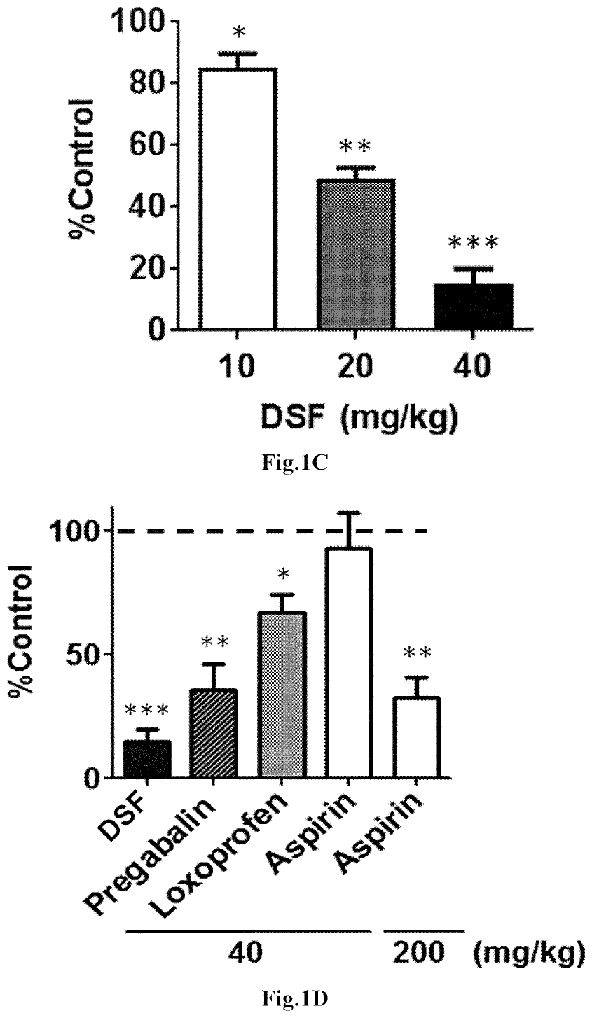

As a result, a reduction in pain threshold associated with sciatic nerve ligation (CCI) was observed, and the reduction in pain threshold was prevented to a significant degree by administration of DSF at a dose of 40 mg/kg (FIG. 2; ** indicates p<0.01). The result indicates that DSF exhibits excellent analgesic effects on neuropathic pain.

### 3. Evaluation of Analgesic Effects of a Metal Complex of Diethyldithiocarbamate by Formalin Test

DSF is metabolized in the body to generate diethyldithiocarbamate (DDC). The analgesic effects of metal complexes of DDC, which are considered to generate DDC in the body as well as DSF, was evaluated by the formalin test. The formalin test was performed in the same manner as in the above-described section 1. except that a copper complex of DDC (Cu(DDC)₂) and an iron(III) complex of DDC (Fe(DDC)₃) were used as test drugs. Either of the complexes was administered to mice at a dose of 40 mg/kg.

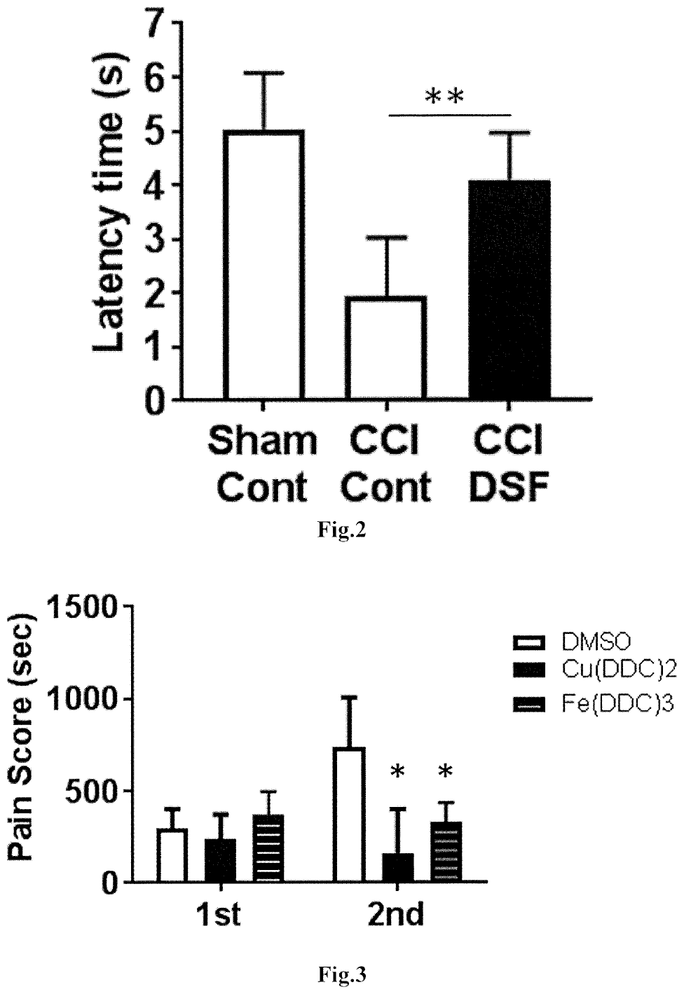

The results are shown in FIG. 3. The metal complexes of DDC were observed to have effects to suppress the second response (* indicates a significant difference at p<0.05 from the solvent control administration group). Moreover, a sedative effect was observed in the mice treated with the complexes, similarly to those administered with DSF.

1. A method for analgesia or sedation, comprising administering an effective amount of any of the following (1) to (5) to a subject:
(1) disulfiram;
(2) a metal complex of diethyldithiocarbamate;
(3) a disulfide capable of generating diethyldithiocarbamate in the body;
(4) a pharmaceutically acceptable salt of (1), (2), or (3); and
(5) a solvate of (1), (2), (3), or (4).

2. The method according to claim 1, wherein the metal complex is a complex of a divalent or higher valent metal.

3. The method according to claim 1, wherein the pain to be relieved is a neuropathic pain.

4. The method according to claim 2, wherein the pain to be relieved is a neuropathic pain.
